(19) United States
(12) Patent Application Publication
(10) Pub. No.: US 2026/0184723 A1
(43) Pub. Date: Jul. 2, 2026

(54) NEW OVATODIOLIDE DERIVATIVES EFFECTIVE FOR TREATING AUTOIMMUNE DISEASES

(71) Applicant: ARJIL BIOTECH HOLDING COMPANY LIMITED, Hsinchu City (TW)

(72) Inventors: Yeh B WU, Hsinchu City (TW); Hui Ju LIANG, Hsinchu City (TW); On LEE, Hsinchu City (TW); Wan-Hsu YANG, Hsinchu City (TW)

(73) Assignee: ARJIL BIOTECH HOLDING COMPANY LIMITED, Hsinchu City (TW)

(21) Appl. No.: 19/436,236

(22) Filed: Dec. 30, 2025

Related U.S. Application Data
(60) Provisional application No. 63/740,640, filed on Dec. 31, 2024.

(51) Int. Cl. C07D 493/08; A61K 31/365; A61P 1/16; A61P 17/06

(52) U.S. Cl. CPC C07D 493/08; A61K 31/365; A61P 1/16; A61P 17/06

(57) ABSTRACT

The present invention pertains to method or pharmaceutical composition of new compounds, AR100-RA1-Cys and AR100-RA1-ST, which have unexpectedly effects in treating auto-immune disease caused by hepatocellular inflammation or psoriasis. Also provided are the preparation processes of the compounds.

### FIELD OF THE INVENTION

The invention relates to new ovatodiolide derivatives, which is characterized by the efficacy in treating an auto-immune disease.

### BACKGROUND OF THE INVENTION

Auto-immune diseases are a group of disorders characterized by the immune system mistakenly attacking the body's own tissues. This occurs when the immune system, which typically defends against harmful pathogens, begins to identify the body's healthy cells as foreign invaders. The resulting immune response leads to inflammation, tissue damage, and a wide range of symptoms depending on which part of the body is affected. Auto-immune diseases can be systemic, affecting multiple organs, or localized to specific areas, and they are often chronic, requiring long-term management.

Psoriasis is a chronic, immune-mediated skin condition characterized by the rapid proliferation of skin cells, leading to formation of thick, red plaques typically covered with silvery scales on the skin's surface. This occurs due to an overactive immune response that accelerates the life cycle of skin cells, causing them to accumulate rapidly. The condition typically presents with red, inflamed patches covered with silvery scales, and can cause itching, pain, and discomfort. While psoriasis primarily affects the skin, it is also associated with other serious health conditions, including psoriatic arthritis, cardiovascular disease, and depression. Although its symptoms and recurrence can be managed through various treatments, including topical medications (such as steroids and vitamin D analogs), phototherapy (using ultraviolet light on the affected areas), oral medications (such as methotrexate and cyclosporine), and biologics (injectable drugs targeting specific immune molecules), there is no cure for psoriasis.

Auto-immune hepatitis (AIH), another example of auto-immune diseases, can also be characterized by hepatocellular inflammation. Severe AIH may progress into liver cirrhosis, hepatocellular carcinoma, or even death. There are two types of AIH, and both are female-predominant. The common autoantibodies in type I are antinuclear antibodies (ANAs) and anti-smooth muscle antibodies (ASMAs). Type II is less usual than type I. The common autoantibody in type II is anti-liver kidney microsomal antibody type 1 against cytochrome P450 enzyme CYP2D6 (anti-LKM1). 40% of AIH patients are co-presented with other auto-immune disorders, such as systemic lupus erythematosus, rheumatoid arthritis, and type I diabetes. Standard treatments are steroids and azathioprine, leading to disease remission in 80˜90% of patients. Azathioprine, an immunosuppressant, is usually in conjunction with the use of corticosteroids. Of note, steroid monotherapy also relieves AIH symptoms and improves survival. Dexamethasone, in particular, is a long-acting synthetic corticosteroid. It is often taken as an anti-inflammatory and immunosuppressive medication.

It is still desirable to develop new approach for treating auto-immune diseases.

### SUMMARY OF THE INVENTION

The present invention provides ovatodiolide derivatives, AR100-RA1-Cys and AR100-RA1-ST, which exhibit remarkable effects in treating auto-immune diseases.

In one aspect, the present invention provides a compound, named as AR100-RA1-Cys, which has a structure selected from the group consisting of:

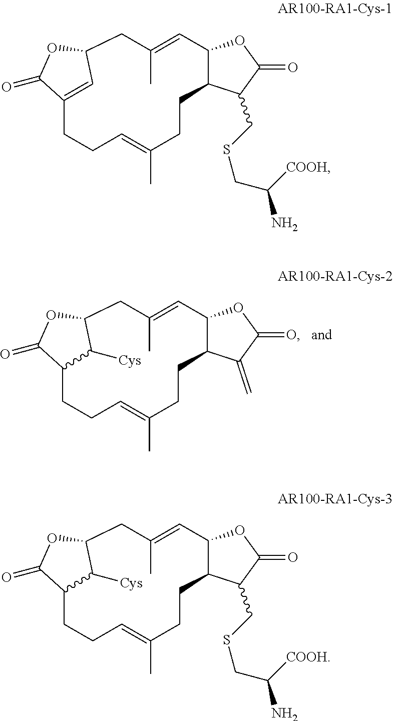

In a further aspect, the present invention provides a compound, named as AR100-RA1-ST, which has a structure selected from the group consisting of:

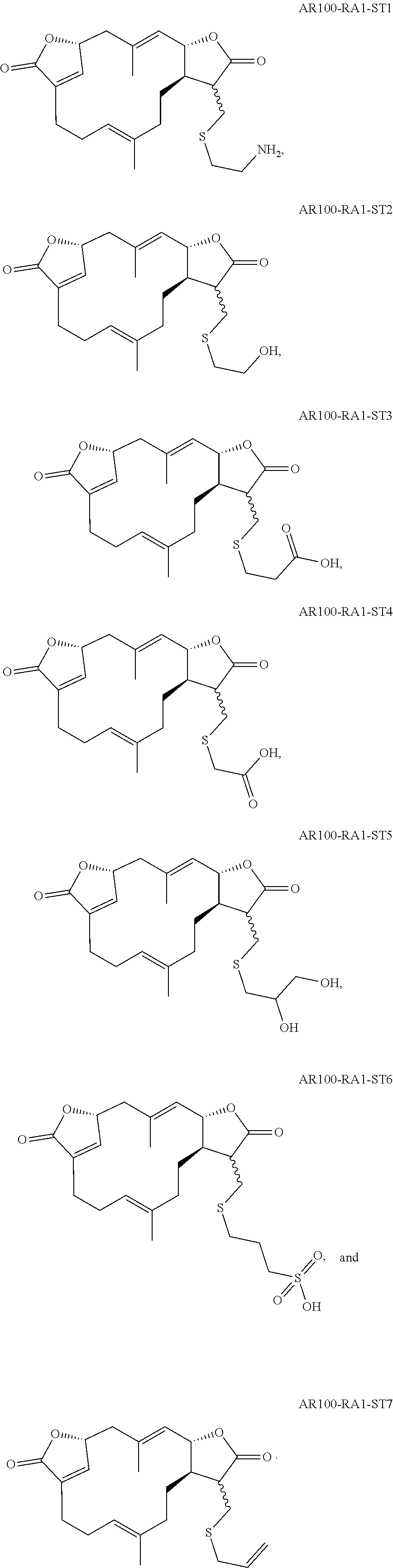

In a further aspect, the present invention provides a method for preparing the compound AR100-RA1-Cys, comprising the steps of:
(A) mixing AR100-RA1 and NaHCO₃ in 75% Ethanol to form a mixture;
(B) adding L-Cysteine Hydrochloride Monohydrate (Cys) into the mixture of step (A);
(C) stirring the mixture of step (B) at around 65° C.;
(D) cooling down the mixture of step (C) to room temperature;
(E) adding HCl into the mixture of step (D) slowly to quench;
(F) filtering and concentrating the mixture of step (E) under reduced pressure; and
(G) purifying the mixture of step (F) to provide AR100-RA1-Cys as light-yellow solid.

In one another aspect, the present invention provides a method for preparing the compound AR100-RA1-ST, comprising the steps of:
(A) mixing AR100-RA1 and NaHCO₃ in 75% Ethanol to form a mixture;
(B) adding one of the 7 compounds as shown below into the mixture of step (A):

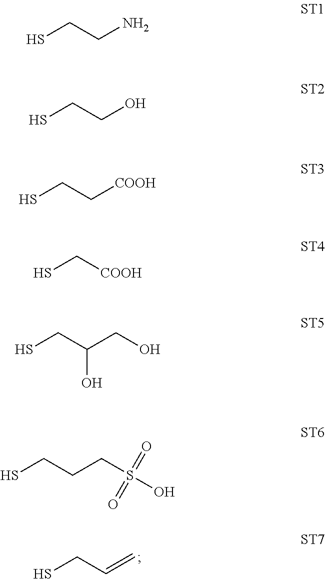

(C) stirring the mixture of step (B) overnight at room temperature;
(D) diluting the mixture of step (C) with water;
(E) adjusting the mixture of step (D) to pH of 2˜4 by adding acid; and
(F) filtering and concentrating the mixture of step (E) to provide AR100-RA1-ST as light-yellow solid.

In a yet aspect, the present invention provides a pharmaceutical composition, which comprises a therapeutically effective amount of AR100-RA1-Cys or AR100-RA1-ST, or combination thereof, together with a pharmaceutically acceptable carrier.

In an embodiment of the invention, the pharmaceutical composition is used for treating an auto-immune disease.

According to the examples of the invention, the auto-immune disease is selected from the group consisting of autoimmune hepatitis, autoimmune pancreatitis, celiac disease, diabetes mellitus type 1, Sjogren' syndrome, ulcerative colitis, Crohn's disease, reflex sympathetic dystrophy, post myocardial infarction syndrome, Graves' disease, inflammatory bowel disease, multiple sclerosis, psoriasis, rheumatoid arthritis, cardiomyopathy and systemic lupus erythematosus.

In a further yet aspect, the present invention provides a method for treating an auto-immune disease in a subject comprising administering to the subject a therapeutically effective amount of the compound according to the invention, or the pharmaceutical composition comprising them.

In one particular example of the invention, the compound AR100-RA1-Cys is AR100-RA1-Cys-1.

In one particular example of the invention, the compound AR100-RA1-ST is AR100-RA1-ST4.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the conditions when verifying the structure of AR100-RA1-Cys by ¹³C NMR.

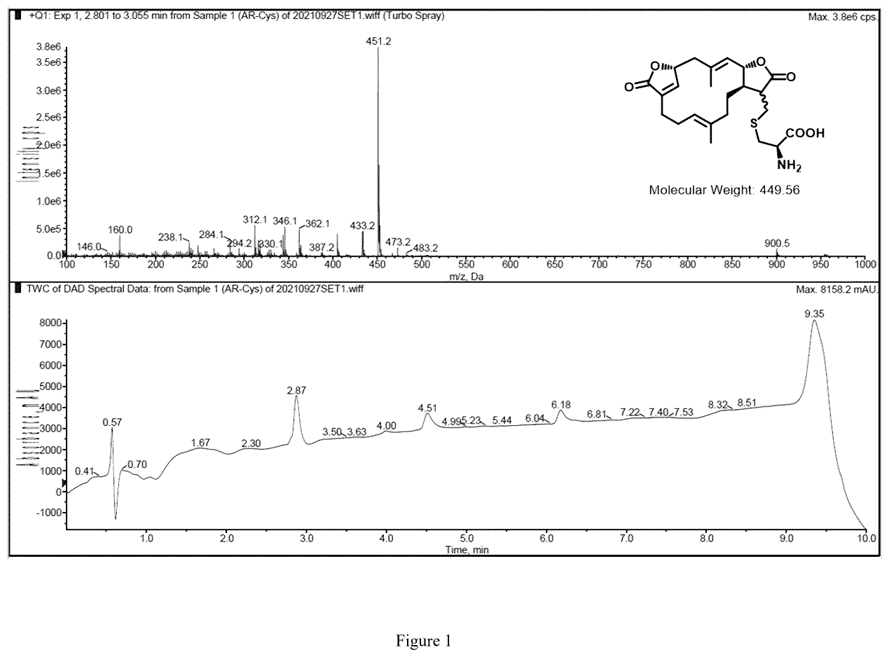

FIG. 2 shows the conditions when verifying the structure of AR100-RA1-Cys by LC-MS.

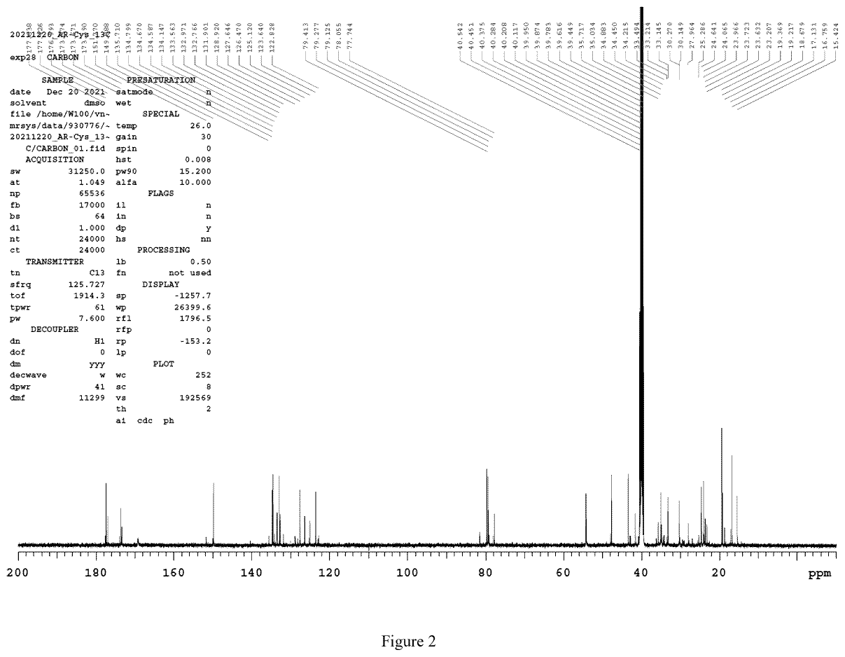

FIG. 3 shows the conditions when verifying the structure of AR100-RA1-ST4 by ¹³C NMR.

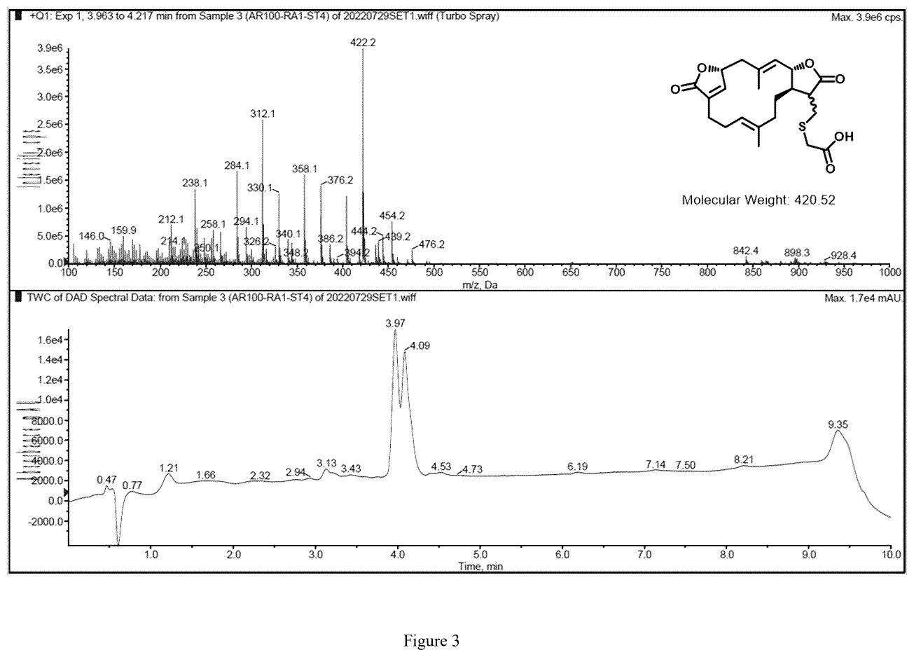

FIG. 4 shows the conditions when verifying the structure of AR100-RA1-ST4 by LC-MS.

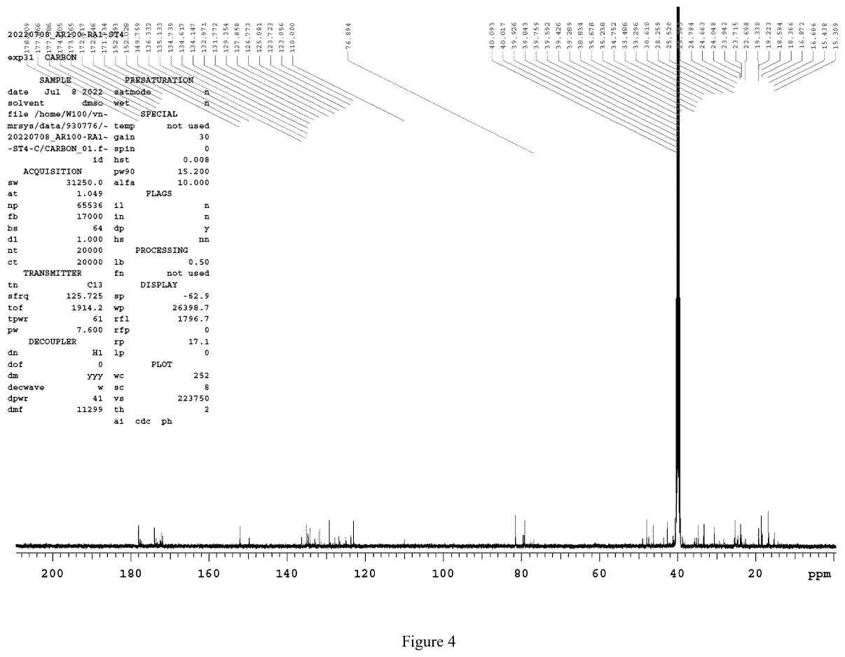

FIG. 5 shows the effect of ovatodiolide derivatives on body weight of Con A-induced acute hepatitis mice.

FIGS. 6A and 6B show the organs weight of Con A-induced acute hepatitis mice; wherein FIG. 6A shows the effect of ovatodiolide derivatives on liver weight; and FIG. 6B shows the effect of ovatodiolide derivatives on spleen weight.

FIGS. 7A and 7B show the serum biochemistry of Con A-induced acute hepatitis mice; wherein FIG. 7A shows the effect of AR100-RA1-Cys and AR100-RA1-ST4 on GOT level; and FIG. 7B shows the effect of AR100-RA1-Cys and AR100-RA1-ST4 on GPT level.

FIG. 8 shows the representative images of the liver section stained with H&E red from a Con A-induced acute hepatitis mice under the effect of test articles.

FIG. 9 shows the effect of AR100-RA1-Cys and AR100-RA1-ST4 on liver histological evaluation of Con A-induced acute hepatitis mice.

FIG. 10 shows the experiment design of IMQ-induced psoriasis model.

FIG. 11 shows the comparison of skin symptoms of BALB/c mice on Day 7 of IMQ-induced psoriasis and the treatment with AR100-RA1-Cys.

FIG. 12 shows the comparison of the back redness score of BALB/c mice on Day 7 of IMQ-induced psoriasis and the treatment with AR100-RA1-Cys.

FIG. 13 shows the comparison of back scaling score of BALB/c mice on Day 7 of IMQ-induced psoriasis and the treatment with AR100-RA1-Cys.

FIG. 14 shows the comparison of back thickness score of BALB/c mice on Day 7 of IMQ-induced psoriasis and the treatment with AR100-RA1-Cys.

FIG. 15 shows the PASI score of BALB/c mice on Day 7 of IMQ-induced psoriasis and the treatment with AR100-RA1-Cys.

FIGS. 16A and 16B shows the results of histopathological analysis of skin sections; wherein FIG. 16A shows the representative images of HE staining; and FIG. 16B shows the quantification result thereof.

FIG. 17 shows the chemical structure of the compound AR100-RA1 (ovatodiolide).

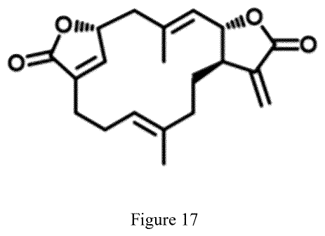

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

As used herein, the term “autoimmune disease” refers to a condition arising from an abnormal immune response to a normal body part. There are at least 80 types of autoimmune diseases. Examples of autoimmune diseases include, but not limited to, autoimmune hepatitis, autoimmune pancreatitis, celiac disease, diabetes mellitus type 1, Sjogren' syndrome, ulcerative colitis, Crohn's disease, reflex sympathetic dystrophy, post myocardial infarction syndrome, Graves' disease, inflammatory bowel disease, multiple sclerosis, psoriasis, rheumatoid arthritis, cardiomyopathy and systemic lupus erythematosus.

The term “pharmaceutically acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, tragacanth gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

According to the invention, the “pharmaceutical composition” may be formulated with a pharmaceutically acceptable carrier by standard or commonly used methods. One example of the pharmaceutically acceptable carrier is hydrogel or water.

The terms “subject”, “individual”, and “patient” are used interchangeably herein to refer to a living animal, including a human and a non-human animal. The “subject” may, for example, be an organism possessing immune cells capable of responding to antigenic stimulation, and stimulatory and inhibitory signal transduction through cell Surface receptor binding. The Subject may be a mammal. Such as a human or non-human mammal, for example, dogs, cats, pigs, cows, sheep, goats, horses, rats, and mice. The term “subject” does not preclude individuals that are entirely normal with respect to a disease, or normal in all respects.

The term “treatment” or” treating” refers to a therapeutic or preventative measure. The treatment may be administered to a subject having a medical disorder or who ultimately may acquire the disorder, in order to prevent, cure, delay, reduce the severity of or ameliorate one or more symptoms of a disorder or recurring disorder, or in order to prolong the survival of a subject beyond that expected in the absence of such treatment.

The term “serum biochemistry” refers to one or more biomarkers measured by biochemical levels in the serum. biochemical levels in the serum can be analyzed by some conventional assays, such as glutamic oxaloacetic transaminase (GOT), glutamic pyruvic transaminase (GPT), alkaline phosphatase (ALP), high-density lipoprotein cholesterol (HDL-C), and total cholesterol (TCHO) analysis. According to the present invention, the compound describe herein can be used to maintain or improve the GOT/GPT value.

The term “liver diseases” refers to liver cell injury or liver damage caused by multiple factors such as NASH (nonalcoholic steatohepatitis), AIH (auto-immune hepatitis), liver cirrhosis, or hepatocellular carcinoma. The present invention proposed the histological evidences to determine the hepatic necrosis score of mice.

The term “therapeutically effective amount” as used herein refers to an amount of a compound or pharmaceutical agent which, as compared to a corresponding subject who has not received such amount, results in an effect in treatment, healing, prevention, or amelioration of a disease, disorder, or side effect, or a decrease in the rate of advancement of a disease or disorder.

According to the invention, the method, use or composition described herein could be administrated a subject in combination with at least one additional therapeutic agent.

In the present invention, the term “ovatodiolide” or “Ova,” which is also called as “AR100-RA1” hereinafter, refers to a compound having a chemical structure as shown below:

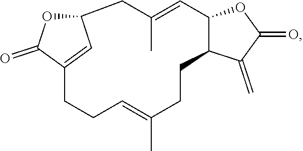 [SMILES: C=C1C(=O)O[C@H]2/C=C(\C)C[C@@H]3C=C(CC/C=C(\C)CC[C@H]12)C(=O)O3]

which is a candidate compound of Arjil pharmaceuticals Co., Ltd., showing extraordinary effects on treating cardiovascular disease and alleviating cytotoxicity, which is illustrated in U.S. Pat. No. >>______.

The present invention provides new ovatodiolide derivatives, and they and their pharmaceutical composition can be used for alleviating auto-immune disease, such as auto-immune hepatitis (AIH) or Psoriasis.

According to the present invention, the ovatodiolide derivatives include AR100-RA1-Cys, also called as “Ova-Cys” and AR100-RA1-ST, also called as “Ova-ST”.

According to the invention, the compound AR100-RA1-Cys is prepared by Michael Addition as shown in the scheme below:

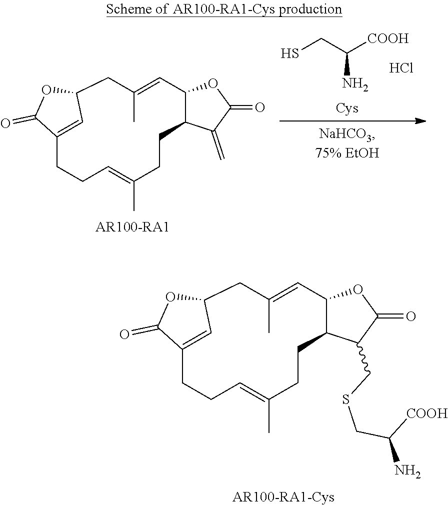

To obtain the light-yellow solid, with 3.91 g and a yielding rate 43.5%, of AR100-RA1-Cys, the AR100-RA1 (6.57 g, 20.0 mmol) was dissolved in 75% EtOH (100 ml). The addition of NaHCO₃ (6.72 g, 80 mmol) and L-Cysteine Hydrochloride Monohydrate (Cys) (3.15 g, 26.0 mmol) were added into the solution. After the mixture solution was stirred at 65° C. for 1.5 hours, followed by TLC measurement, 4N HCl (20 ml, 80.0 mmol) was added slowly into it under room temperature. Then, 50 ml of H2O and MeOH were added to the mixture solution, respectively. The aspirator and dry freezer were used to concentrate and dry the mixture solution after 10 mins of reaction. After adding 10 ml of MeOH, the products were sonicated for 1 min. The process of elimination of the solids and the concentration of the mixture solution by using the aspirator was further conducted. With the DCM/MeOH as the eluent, the final products were purified via silica gel column chromatography.

In a particular example of the invention, AR100-RA1-Cys comprises a cysteine group having the structures as follow:

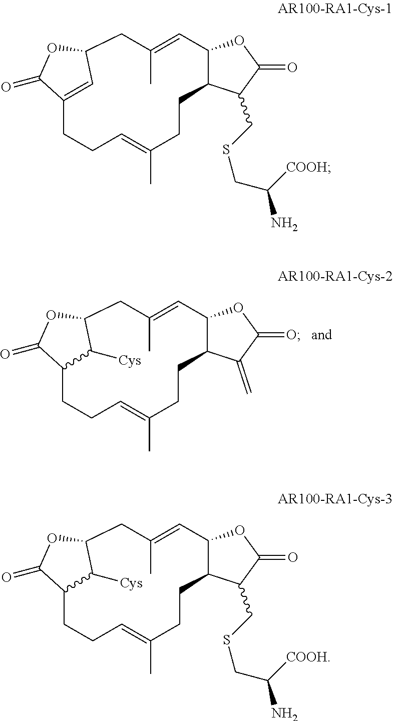

According to the invention, the compound AR100-RA1-ST is prepared by Michael Addition as shown in the scheme below:

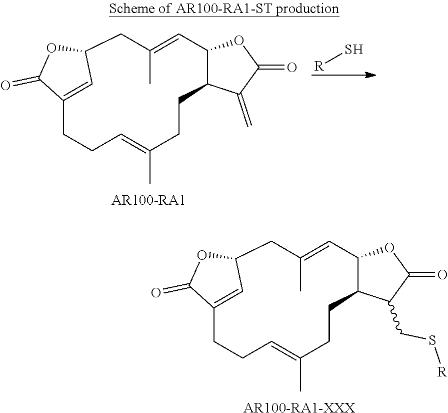

To obtain AR100-RA1-ST1, a mixture of AR100-RA1 (656.8 mg, 2.0 mmol, 1.0 eq) and NaHCO₃ (840.0 mg, 10.0 mmol, 5.0 eq) in 70% EtOH (10 mL) was added 2-Mercaptoethylamine hydrochloride (ST1) (454.4 mg, 4.0 mmol, 2.0 eq). The reaction mixture was stirred overnight at room temperature. After dilution with water (30 mL), the pH of the reaction mixture was adjusted to 3 by the addition of acetic acid. The reaction mixture filtered and concentrated in vacuo to provide AR100-RA1-ST1 (749.6 mg, yield 92.4%) as light-yellow oil. The scheme of AR100-RA1-ST1 is shown as follow:

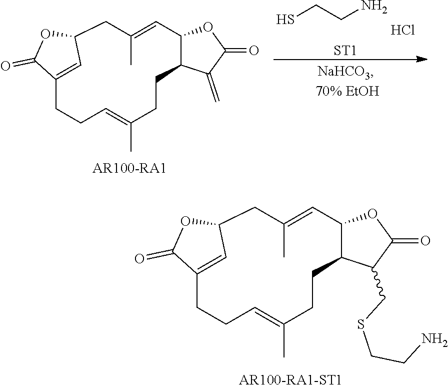

To obtain AR100-RA1-ST2, a mixture of AR100-RA1 (656.8 mg, 2.0 mmol, 1.0 eq) and NaHCO₃ (840.0 mg, 10.0 mmol, 5.0 eq) in 70% EtOH (10 mL) was added 2-Mercaptoethanol (ST2) (312.5 mg, 4.0 mmol, 2.0 eq). The reaction mixture was stirred overnight at room temperature. The reaction mixture filtered and concentrated under reduces pressure. The resultant was purified by preparative high-performance liquid chromatography to provide AR100-RA1-ST2 (256.0 mg, 31.5%) as light-yellow oil. The scheme of AR100-RA1-ST2 is shown as follow:

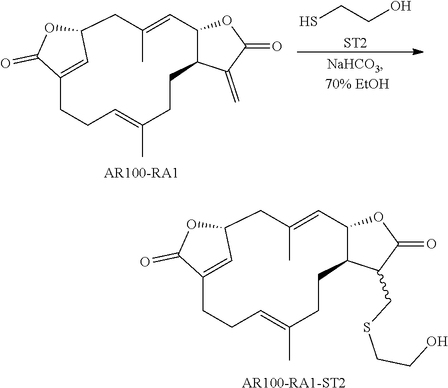

To obtain AR100-RA1-ST3, a mixture of AR100-RA1 (656.8 mg, 2.0 mmol, 1.0 eq) and NaHCO₃ (840.0 mg, 10.0 mmol, 5.0 eq) in 70% EtOH (10 mL) was added 3-Mercaptopropionic acid (ST3) (318.5 mg, 3.0 mmol, 1.5 eq). The reaction mixture was stirred overnight at room temperature. The reaction mixture filtered and concentrated under reduces pressure. The resultant was purified by preparative high-performance liquid chromatography to provide AR100-RA1-ST3 (522.0 mg, 60.0%) as light-yellow solid. The scheme of AR100-RA1-ST3 is shown as follow:

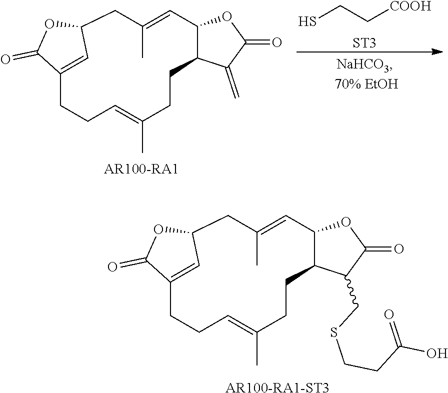

To obtain AR100-RA1-ST4, a mixture of AR100-RA1 (656.8 mg, 2.0 mmol, 1.0 eq) and NaHCO₃ (840.0 mg, 10.0 mmol, 5.0 eq) in 70% EtOH (10 mL) was added with mercaptoacetic acid (ST4) (457.0 mg, 4.0 mmol, 2.0 eq). The reaction mixture was stirred overnight at room temperature. After dilution with water (30 mL), the pH of the reaction mixture was adjusted to 2 by the addition of 2N HCl. The reaction mixture filtered and concentrated in vacuo to provide AR100-RA1-ST4 (714.0 mg, 85.0%) as light-yellow solid. The scheme of AR100-RA1-ST4 is shown as follow:

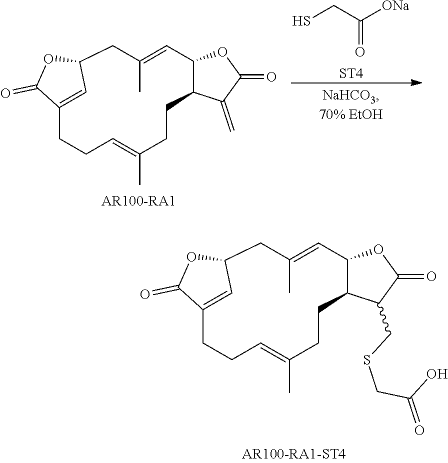

To obtain AR100-RA1-ST5, a mixture of AR100-RA1 (656.8 mg, 2.0 mmol, 1.0 eq) and NaHCO₃ (840.0 mg, 10.0 mmol, 5.0 eq) in 70% EtOH (10 mL) was added 3-Mercapto-1,2-propanediol (ST5) (433.0 mg, 4.0 mmol, 2.0 eq). The reaction mixture was stirred overnight at room temperature. After dilution with water (30 mL), the pH of the reaction mixture was adjusted to 3 by the addition of acetic acid. The reaction mixture filtered and concentrated in vacuo to provide AR100-RA1-ST5 (716.0 mg, 82.0%) as light-yellow oil. The scheme of AR100-RA1-ST5 is shown as follow:

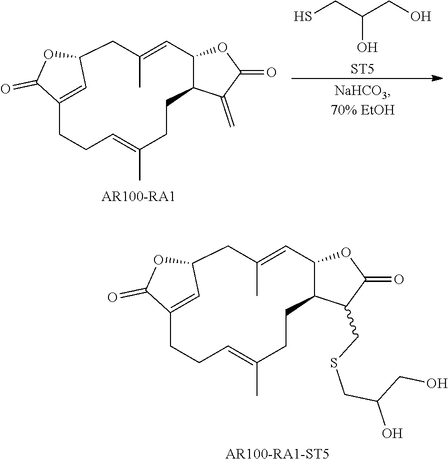

To obtain AR100-RA1-ST6, a mixture of AR100-RA1 (656.8 mg, 2.0 mmol, 1.0 eq) and NaHCO₃ (840.0 mg, 10.0 mmol, 5.0 eq) in 70% EtOH (10 mL) was added Sodium 3-Mercapto-1-propanesulfonate (ST6) (713.0 mg, 4.0 mmol, 2.0 eq). The reaction mixture was stirred overnight at room temperature. After dilution with water (30 mL), the pH of the reaction mixture was adjusted to 3 by the addition of acetic acid. The reaction mixture filtered and concentrated in vacuo to provide AR100-RA1-ST6 (765.7 mg, 79.0%) as light-yellow oil. The scheme of AR100-RA1-ST6 is shown as follow:

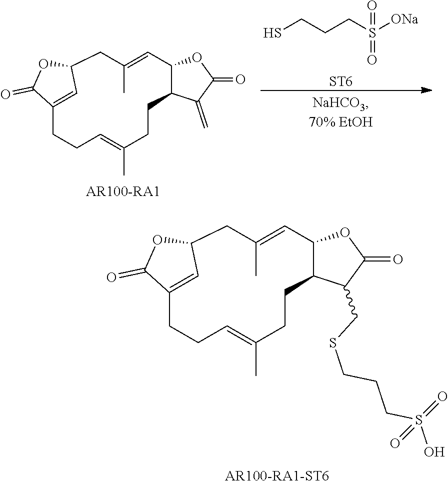

To obtain AR100-RA1-ST7, a mixture of AR100-RA1 (656.8 mg, 2.0 mmol, 1.0 eq) and NaHCO₃ (840.0 mg, 10.0 mmol, 5.0 eq) in 70% EtOH (10 mL) was added Ally mercaptan (ST7) (713.0 mg, 4.0 mmol, 2.0 eq). The reaction mixture was stirred overnight at room temperature. The reaction mixture filtered and concentrated under reduces pressure to provide AR100-RA1-ST7 (587.7 mg, 73.0%) as light-yellow oil. The scheme of AR100-RA1-ST7 is shown as follow:

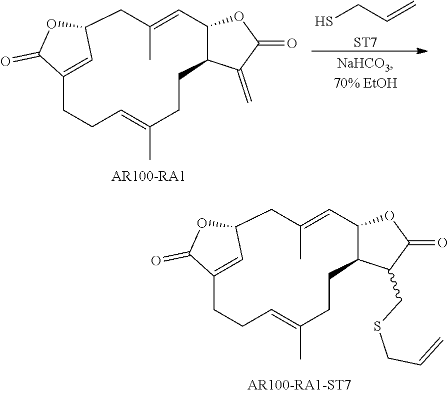

In one example of the present invention, C57BL/6 mice was induced to present acute hepatitis by intravenous injection of concanavalin A (Con A), which is a widely-used strategy to study T cell-mediated hepatitis. Con A is a lectin that can activate CD4+ T cells, produce cytokines, and lead to liver cell damage. Thus, pro-inflammatory cytokines such as interferon (IFN)-γ, tumor necrosis factor (TNF)-α, and interleukin (IL)-6 may play a significant role in hepatitis.

According to the physiological conditions including body weight, liver weight, spleen weight, serum biochemistry, and liver histopathology of Con A-induced acute hepatitis in C57BL/6 mice, it was reported that AR100-RA1-Cys and AR100-RA1-ST are remarkable compounds for treating auto-immune hepatitis (AIH). The results specifically indicated improvements in GOT/GPT values and hepatocyte necrosis levels upon administration of AR100-RA1-Cys and AR100-RA1-ST.

Moreover, AR100-RA1-Cys is ascertained to have alleviating effect on IMQ-induced psoriasis by evaluating Psoriasis Area and Severity Index Score (PASI Score), which includes the evaluation based on three main psoriasis symptoms: erythema (back redness), desquamation (back scaling), and induration (back thickness), and histopathological analysis.

The present invention provides a method and pharmaceutical composition for improving auto-immune diseases such as auto-immune hepatitis (AIH) or psoriasis, comprising administrating AR100-RA1-Cys or AR100-RA1-ST as effective compounds to a subject in need thereof.

The present invention is further illustrated by the following examples, which are provided for the purpose of demonstration rather than limitation.

### EXAMPLES

### Materials and Methods

### AR100-RA1-Cys (Ova-Cys) Preparation

To a mixture of AR100-RA1 (6.57 g, 20.0 mmol, 1.0 eq) and NaHCO₃ (6.72 g, 80.0 mmol, 4.0 eq) in 75% Ethanol (EtOH) (100 mL) was added L-Cysteine Hydrochloride Monohydrate (Cys) (3.15 g, 26.0 mmol, 1.3 eq). The reaction mixture was stirred at 65° C. for 1.5 hours. The reaction was cooled down to the room temperature, 4N HCl (20 mL, 80.0 mmol, 4.0 equiv) was added slowly to quench. The reaction mixture filtered and concentrated under reduces pressure. The resultant was purified by silica gel column chromatography eluting with Diclomethane (DCM)/Methanol (MeOH) (10/1-8/1 (v/v)) to provide AR100-RA1-Cys (3.91 g, yield 43.5%) as light-yellow solid.

### 2. AR100-RA1-ST4 (Ova-ST) Preparation

To a mixture of AR100-RA1 (656.8 mg, 2.0 mmol, 1.0 eq) and NaHCO₃ (840.0 mg, 10.0 mmol, 5.0 eq) in 70% EtOH (10 mL) was added mercaptoacetic acid (ST4) (457.0 mg, 4.0 mmol, 2.0 eq). The reaction mixture was stirred overnight at room temperature. After dilution with water (30 mL), the pH of the reaction mixture was adjusted to 2 by the addition of 2N HCl. The reaction mixture filtered and concentrated in vacuo to provide AR100-RA1-ST4 (714.0 mg, 85.0%) as light-yellow solid.

### 3. Animals Preparation

6˜8-week-old male C57BL/6 mice (Auto-immune hepatis model) or BALB/c mice (psoriasis model) were purchased from National Laboratory Animal Center (NLAC), Taiwan or BioLASCO Taiwan Co., Ltd. Animals were housed in the Animal Laboratory for Biomedical Research in ITRI within conventional cages under standardized conditions (23±2° C., 40˜70% relative humidity, positive pressure, ventilation rate 15˜20 changes per hour, and a 12 hours light/12 hours dark cycle where the dark phase was from 7:30 μm to 7:30 am). Mice were allowed to acclimatize to the new environment for at least one week and fed with a standard chow diet and water ad libitum before the experiment.

### Auto-Immune Hepatis Model

In auto-immune hepatis model, Con A was applied to C57BL/6 mice to mimic the characteristics of human AIH. The experiment was designed as: Group 1 was not administered with Con A as Naïve. Groups 2˜6 were treated with Con A to induce acute hepatitis. Group 2 was orally administered (p.o.) with 10% DMSO (Sigma D5879) and 10% Cremophor EL (Sigma C5135), which dissolved the test articles as Vehicle. Group 3 was orally administered (p.o.) with 0.1 mg/kg of dexamethasone as a benchmark control. Groups 4˜ 6 were orally administered (p.o.) with 50 mg/kg of AR100-RA1, AR100-RA1-Cys, and AR100-RA1-ST4, respectively, where p.o. stands for oral gavage. The dose, expressed as the weight of test articles (mg) per body weight (kg) of test animals, was applied in volumes of 10 mL per kg body weight. All six groups of the experiment are listed as table 1 as below:

TABLE 1
Group design for auto-immune hepatis model

| | | Dose | | |
|---|---|---|---|---|
| Group | Treatment | (mg/kg) | Route | n |
| 1 | Naive | — | — | 3 |
| 2 | Vehicle | 0 | p.o. | 10 |
| 3 | Dexamethasone | 0.1 | p.o. | 10 |
| 4 | AR100-RA1 | 50 | p.o. | 10 |
| 5 | AR100-RA1-Cys | 50 | p.o. | 10 |
| 6 | AR100-RA1-ST4 | 50 | p.o. | 10 |

### Psoriasis Model

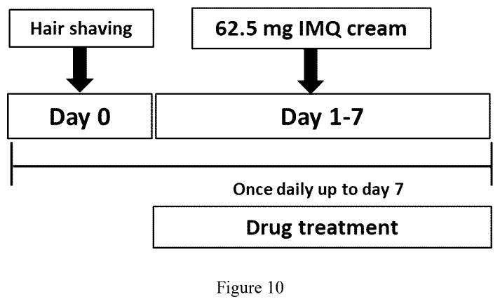

As shown in FIG. 10, in psoriasis model, imiquimod (IMQ) is applied to BALB/c mice to induce psoriasis to mimic the characteristics of human psoriasis. The experiment was designed as: Group 1 was Naïve control. Group 2 was induced with 62.5 mg IMQ administrating on the back. Group 3 was induced with 62.5 mg IMQ administrating on the back and treated with Daivobet after 4 hours. Group 4 to Group 9 were induced with 62.5 mg IMQ administrating on the back and treated with 100 μL AR100-RA1 (50, 100 mg/kg), AR100-RA1-Cys (50, 100 mg/kg), and Zerumbone (50, 100 mg/kg) respectively, after 4 hours. The dose, expressed as the weight of test articles (mg) per body weight (kg) of test animals, was applied in volumes of 10 mL per kg body weight, and where p.o. stands for oral gavage. During this period, the mice's weight, skin redness, swelling and desquamation were observed and scored. All mice were sacrificed 24 hours after the last treatment in the experiment. Skin tissue was collected for pathological analysis and cytokine expression analysis. All groups of the experiment are listed as table 2 below:

TABLE 2
Group design for auto-immune psoriasis model

| | | Dose | | |
|---|---|---|---|---|
| Group | Treatment | (mg/kg) | Route | n |
| 1 | Naive | — | — | 3 |
| 2 | IMQ | — | topical use | 3 |
| 3 | IMQ + Positive | — | topical use | 3 |
| 4 | IMQ + AR100-RA1 | 50 mg/kg | p.o. | 3 |
| 5 | IMQ + AR100-RA1 | 100 mg/kg | p.o. | 3 |
| 6 | IMQ + AR100-RA1-Cys | 50 mg/kg | p.o. | 3 |
| 7 | IMQ + AR100-RA1-Cys | 100 mg/kg | p.o. | 3 |
| 8 | IMQ + Zerumbone | 50 mg/kg | p.o. | 3 |
| 9 | IMQ + Zerumbone | 100 mg/kg | p.o. | 3 |

### 5. Serum Biochemistry

Upon collection, all blood samples were allowed for coagulation for at least one hour. Serum was then obtained at room temperature via 10 minutes of 6,000 rpm centrifugation. The biochemical analysis including aspartate aminotransferase (AST/GOT), alanine aminotransferase (ALT/GPT), alkaline phosphatase (ALP), high-density lipoprotein cholesterol (HDL-C), and total cholesterol (TCHO) was measured by an automated clinical chemistry analyzer (FUJI DRI-CHEM 4000i).

### 6. Liver Histopathology

Liver tissues were fixed in 10% neutral buffered formalin, trimmed, embedded in paraffin, sectioned, and stained with hematoxylin and eosin (H&E) or Sirius Red. Quantitative analysis of the stained sections was examined microscopically under the optical microscope (Leica DM2700M, USA). The severity grading system for microscopic liver lesions is described by Knodell et al. (1981), Ruwart et al. (1989), and the literature published in Toxicologic Pathology (Shackelford et al., 2002). Both the staining and histological examination were conducted by an experienced veterinarian pathologist.

### 7. Psoriasis Area and Severity Index Score, PASI Score

In the experiment, the primary evaluation was based on three main psoriasis symptoms: erythema (back redness), desquamation (back scaling), and induration (back thickness). Each symptom was scored on a scale from 0 to 4, where 0 indicates no symptoms, 1 indicates mild symptoms, 2 indicates moderate symptoms, 3 indicates severe symptoms, and 4 indicates very severe symptoms. The total possible score is 16. Assessments were conducted twice weekly (van der Fits, Mourits et al. 2009; Luo, Wu et al. 2016).

### 8. Histopathological Analysis of Skin Sections

Histopathological analysis was performed using hematoxylin and eosin (H&E) staining. Hematoxylin will stain cell nuclei blue, while eosin will stain the cytoplasm pink. Under the microscope, changes in the stratum corneum, epidermis, and dermis of psoriatic skin will be analyzed (Luo, Wu et al. 2016).

### 9. Statistical Analysis

### Auto-Immune Hepatis Model

All data are presented as mean±SEM. To determine the effect of test articles on body weight, liver weight, spleen weight, kidney weight, and serum biochemistry, Student's t-test was performed. To evaluate the effect of test articles on liver histopathology, the Mann-Whitney test was performed. A p-value less than 0.05 is considered statistically significant.

### Psoriasis Model

Statistical analyses were performed using GraphPad Prism version 9.0. All experimental data are presented as mean±standard deviation. One-way ANOVA or two-way ANOVA was used to compare data between all groups and the IMQ group, followed by Dunnett's multiple comparisons test. A p-value less than 0.05 is considered statistically significant (* p<0.05, **p<0.01, ***p<0.001)

### Results

### Example 1

### Effects on Body Weight in AIH Model

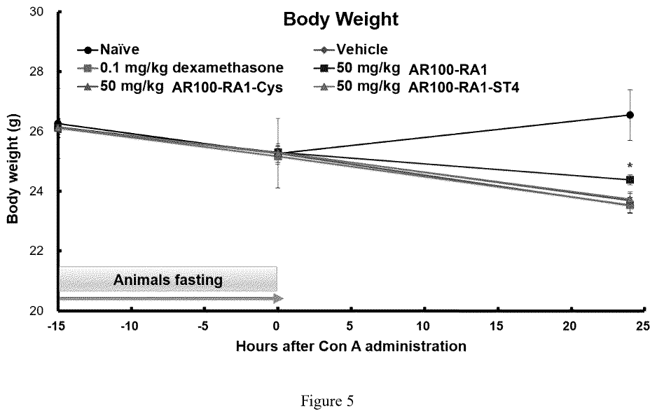

Intravenously injected C57BL/6 mice with Con A resulted in reduced body weight, while the animals receiving 50 mg/kg AR100-RA1, on the other hand, exhibited significantly less body weight decrease as shown in the table below, and could be further illustrated by FIG. 5. The dosing of test articles was 30 min before, 4 hours, and 8 hours after Con A administration. n=3 for Naïve; n=10 for Vehicle, dexamethasone (0.1 mg/kg), AR100-RA1 (50 mg/kg), AR100-RA1-Cys (50 mg/kg), and AR100-RA1-ST4 (50 mg/kg). * p<0.05 vs Vehicle at the same time point using Student's t-test.

| | Body weight (after Con A administration) | | |
|---|---|---|---|
| Group | −15 hr | 0 hr | 24 hr |
| Naive | 26.26 ± 1.17 | 25.27 ± 1.16 | 26.55 ± 0.84 |
| Vehicle | 26.11 ± 0.32 | 25.27 ± 0.31 | 23.52 ± 0.26 |
| 0.1 mg/kg dexamethasone | 26.11 ± 0.31 | 25.17 ± 0.27 | 23.54 ± 0.25 |
| 50 mg/kg AR100-RA1 | 26.15 ± 0.26 | 25.29 ± 0.22 | 24.38 ± 0.17* |
| 50 mg/kg AR100-RA1-Cys | 26.13 ± 0.23 | 25.30 ± 0.22 | 23.69 ± 0.23 |
| 50 mg/kg AR100-RA1-ST4 | 26.13 ± 0.22 | 25.29 ± 0.20 | 23.73 ± 0.24 |

### Example 2

### Effects on Spleen Weight and Liver Weight in AIH Model

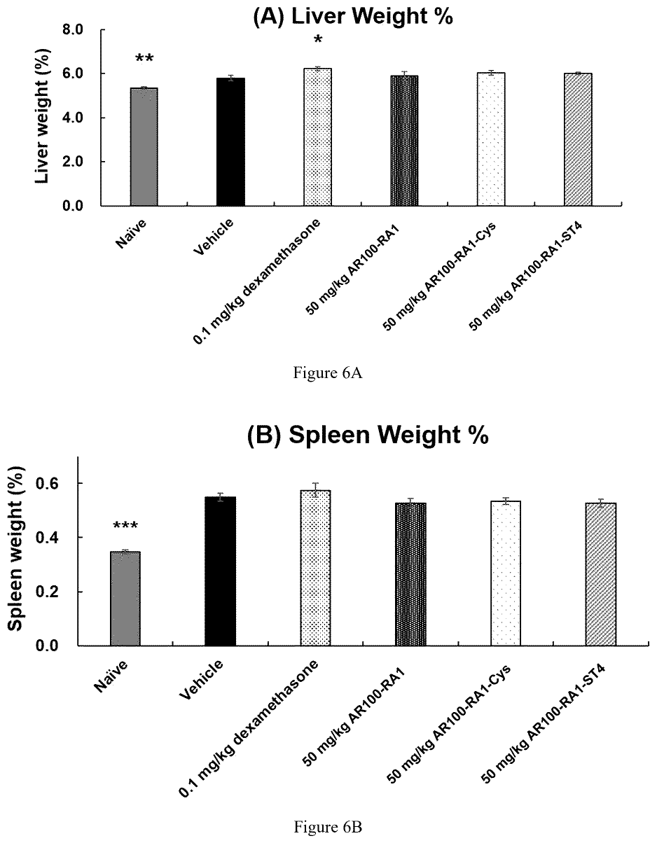

According to the table below, after Con A induction, % liver and % spleen weights were markedly elevated. Moreover, when dosing 0.1 mg/kg dexamethasone, a significantly more increase in % liver weight was observed compared to the Vehicle group. In order to analyze and compare the difference between different groups, the below table could be further illustrated by FIG. 6A and FIG. 6B. The dosing of test articles was 30 min before, 4 hours, and 8 hours after Con A administration. n=3 for Naïve; n=10 for Vehicle, dexamethasone (0.1 mg/kg), AR100-RA1 (50 mg/kg), AR100-RA1-Cys (50 mg/kg), and AR100-RA1-ST4 (50 mg/kg). Organ weight %=organ weight (g)/body weight (g). *p<0.05, **p<0.01, ***p<0.001 vs Vehicle using Student's t-test.

| | Liver weight | | Spleen weight | Spleen weight |
|---|---|---|---|---|
| Group | (g) | Liver weight % | (g) | % |
| Naive | 1.418 ± 0.038 | 5.34 ± 0.05** | 0.092 ± 0.003*** | 0.35 ± 0.01*** |
| Vehicle | 1.363 ± 0.039 | 5.79 ± 0.12 | 0.129 ± 0.004 | 0.55 ± 0.02 |
| 0.1 mg/kg dexamethasone | 1.464 ± 0.032 | 6.21 ± 0.09* | 0.135 ± 0.006 | 0.57 ± 0.03 |
| 50 mg/kg AR100-RA1 | 1.436 ± 0.045 | 5.90 ± 0.20 | 0.129 ± 0.005 | 0.53 ± 0.02 |
| 50 mg/kg AR100-RA1-Cys | 1.426 ± 0.021 | 6.02 ± 0.10 | 0.126 ± 0.003 | 0.53 ± 0.01 |
| 50 mg/kg AR100-RA1-ST4 | 1.423 ± 0.022 | 5.99 ± 0.05 | 0.125 ± 0.004 | 0.53 ± 0.02 |

### Example 3

### Serum Biochemistry in AIH Model

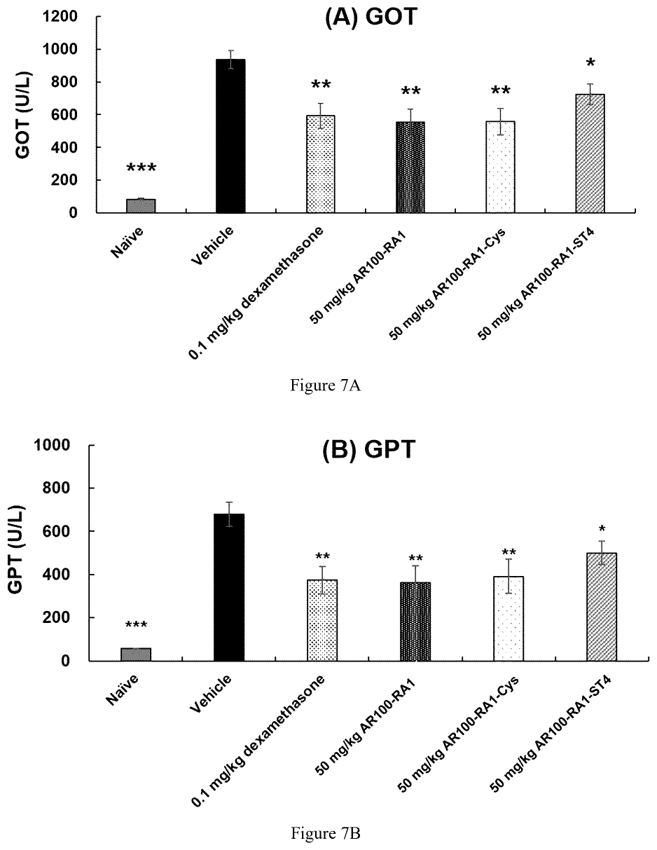

GOT and GPT levels, the biomarkers of liver injury, were significantly elevated in Con A-treated mice compared to untreated Naive as shown in the table below. Compared to the Vehicle group, the GOT and GPT levels were both markedly reduced in the 50 mg/kg AR100-RA1, 50 mg/kg AR100-RA1-Cys, and 50 mg/kg AR100-RA1-ST4 groups, following the dosing of 0.1 mg/kg dexamethasone. In order to analyze and compare the difference between different groups, the below table could be further illustrated by FIG. 7A and FIG. 7B. The dosing of test articles was 30 min before, 4 hours, and 8 hours after Con A administration. n=3 for Naïve; n=10 for Vehicle, dexamethasone (0.1 mg/kg), AR100-RA1 (50 mg/kg), AR100-RA1-Cys (50 mg/kg), and AR100-ST4 (50 mg/kg). GOT, aspartate transaminase. GPT, alanine transaminase. * p<0.05, ** p<0.01, *** p<0.001 vs Vehicle using Student's t-test.

| Group | GOT (U/L) | GPT (U/L) |
|---|---|---|
| Naive | 84.00 ± 7.64*** | 56.33 ± 2.96*** |
| Vehicle | 935.00 ± 56.12 | 679.50 ± 56.27 |
| 0.1 mg/kg dexamethasone | 592.40 ± 76.89** | 373.00 ± 62.72** |
| 50 mg/kg AR100-RA1 | 553.10 ± 81.03** | 362.90 ± 77.43** |
| 50 mg/kg AR 100-RA1-Cys | 557.40 ± 81.15** | 391.40 ± 79.83** |
| 50 mg/kg AR100-RA1-ST4 | 723.90 ± 63.87* | 500.00 ± 54.82* |

### Example 4

### Liver Histopathology in AIH Model

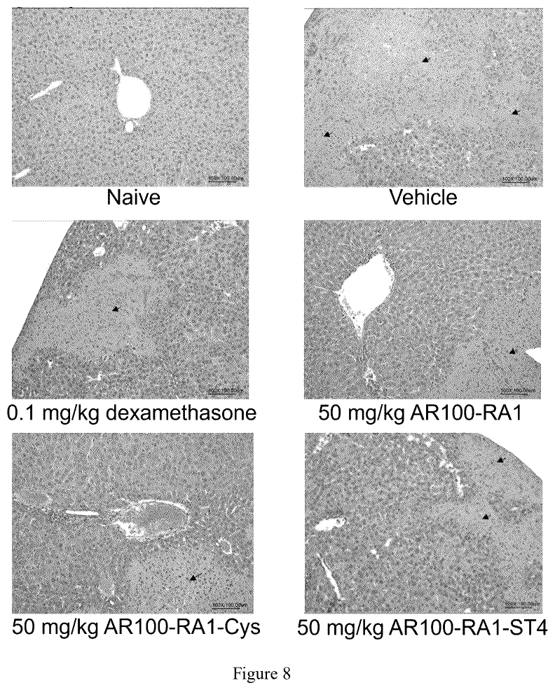

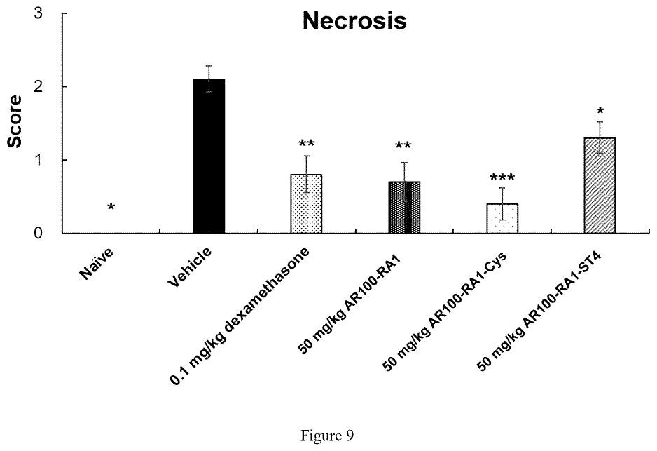

Representative images of the liver section stained with H&E red are presented in FIG. 8. The dosing of test articles was 30 min before, 4 hours, and 8 hours after Con A administration. n=3 for Naïve; n=10 for Vehicle, dexamethasone (0.1 mg/kg), AR100-RA1 (50 mg/kg), AR100-RA1-Cys (50 mg/kg), and AR100-RA1-ST4 (50 mg/kg). The arrows refer to focal hepatocyte necrosis.in Con A-challenged mice is characterized by minimal to mild focal necrosis. According to FIG. 9, 0.1 mg/kg dexamethasone, 50 mg/kg AR100-RA1, 50 mg/kg AR100-RA1-Cys, and 50 mg/kg AR100-RA1-ST4 administration relieved necrosis significantly. The dosing of test articles was 30 min before, 4 hours, and 8 hours after Con A administration. n=3 for Naïve; n=10 for Vehicle, dexamethasone (0.1 mg/kg), AR100-RA1 (50 mg/kg), AR100-RA1-Cys (50 mg/kg), and AR100-RA1-ST4 (50 mg/kg). *p<0.05, **p<0.01, ***p<0.001 vs Vehicle using Mann-Whitney test. The original data are shown in the table below.

| Group | Necrosis |
|---|---|
| Naïve | 0.00 ± 0.00* |
| Vehicle | 2.10 ± 0.18 |
| 0.1 mg/kg dexamethasone | 0.80 ± 0.25** |
| 50 mg/kg AR100-RA1 | 0.70 ± 0.26** |
| 50 mg/kg AR 100-RA1-Cys | 0.40 ± 0.22*** |
| 50 mg/kg AR 100-RA1-ST4 | 1.30 ± 0.21* |

### Example 5

### Effect on Symptoms of Imiquimod (IMQ)-Induced Psoriasis

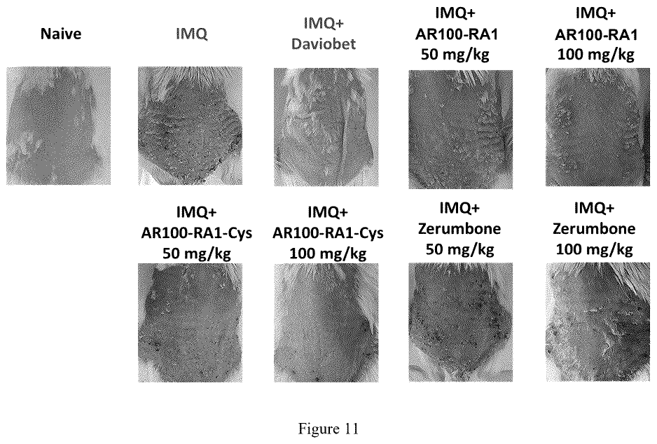

In the IMQ-induced psoriasis model of BALB/c mice over a 7-day period, AR100 series samples, including AR100-RA1, AR100-RA1-Cys and Zerumbone, were administered individually to the mice. As shown in FIG. 11, the clinical features induced by IMQ were alleviated following treatment with AR100 series samples, with a reduction in skin lesions. Based on the reduction in skin lesions, the therapeutic efficacy was ranked from most to least effective as follows: Naïve>Daviobet>AR100-RA1-Cys>AR100-RA1=Zerumbone>IMQ.

### Example 6

### Psoriasis Area and Severity Index Score, PASI Score

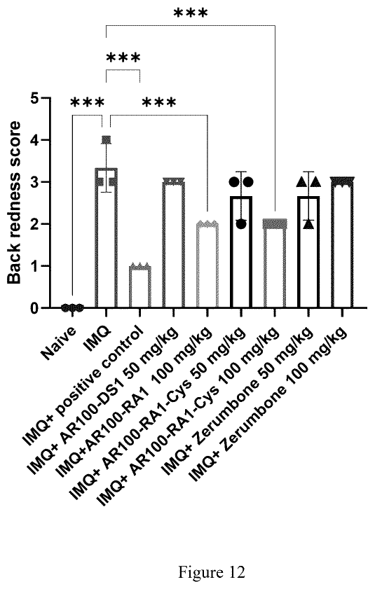

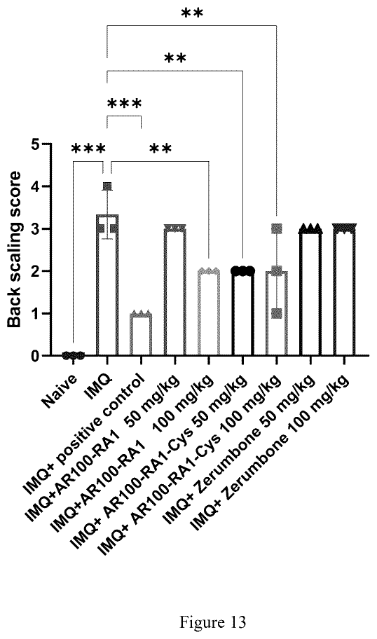

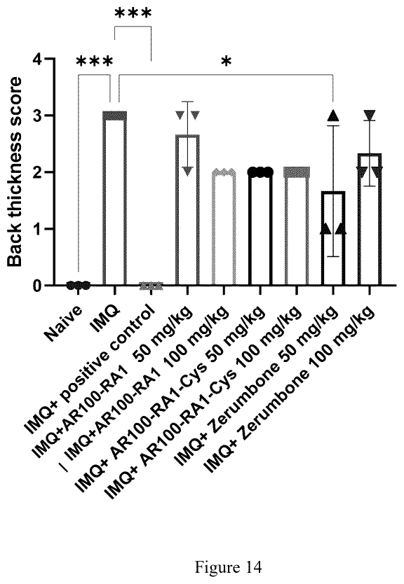

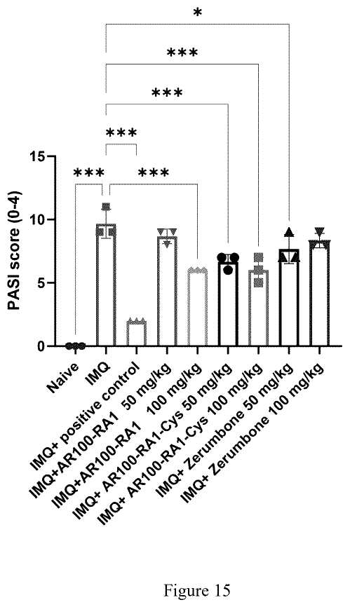

In the IMQ-induced psoriasis model of BALB/c mice, the PASI score of three IMQ symptoms: erythema (back redness), desquamation (back scaling), and induration (back thickness) was recorded, each symptom was scored up to 4 points. As shown in FIG. 12, comparing to the IMQ group on day 7 of the experiment, there was a significant improvement in back redness following treatment with AR100-RA1 (100 mg/kg), AR100-RA1-Cys (100 mg/kg), and the positive control (Daviobet). AR100-RA1 (50 mg/kg), AR100-RA1-Cys (50 mg/kg), and Zerumbone showed no statistically significant in erythema. FIG. 13 shows the reduction of back scaling, treatment with AR100-RA1 (100 mg/kg), AR100-RA1-Cys, and the positive control (Daviobet) led to a significant improvement. As shown in FIG. 14, significant improvement in skin thickness was observed with the positive control (Daviobet), while AR100-RA1 and AR100-RA1-Cys did not significantly reduced skin thickness. As shown in FIG. 15, the therapeutic efficacy based on PASI score was ranked from best to worst as follows: Naïve>Daviobet>AR100-RA1-Cys>AR100-RA1=Zerumbone>IMQ. These results confirm that among the AR100 series samples, AR100-RA1 (100 mg/kg), AR100-RA1-Cys, and Zerumbone (50 mg/kg) effectively alleviate skin symptoms of IMQ-induced psoriasis model.

### Example 7

### Histopathological Analysis of Skin Sections

It was confirmed that IMQ-induced psoriasis model would present the skin desquamation. As shown in FIG. 11, treatment with the positive control (Daviobet), AR100-RA1 (100 mg/kg), and AR100-RA1-Cys obviously reduced the symptom of skin desquamation.

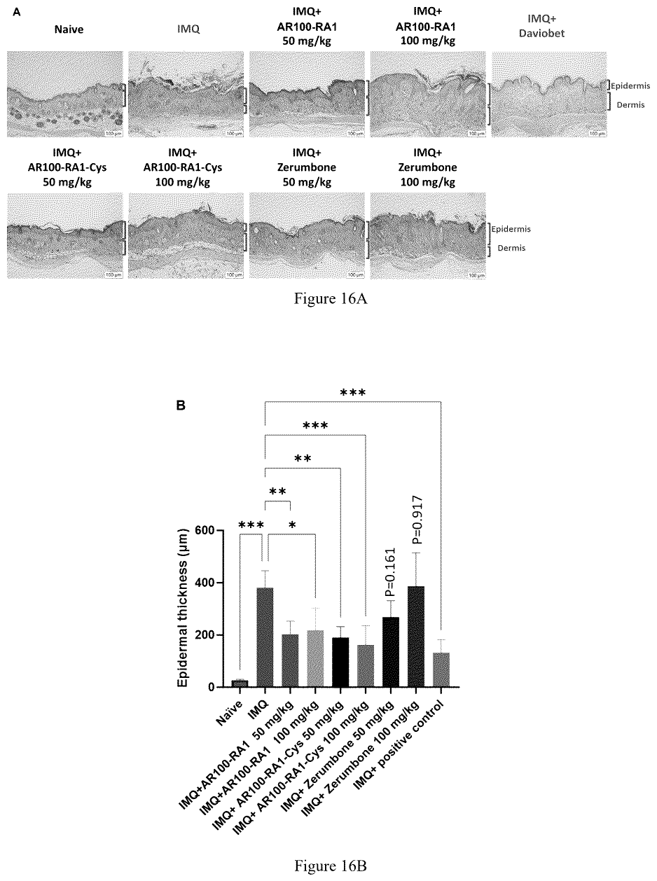

Additionally, skin thickening was observed following IMQ induction, prompting further analysis through histopathological examination. The structure of the epidermis and dermis was observed using HE staining. As shown in FIGS. 16A and 16B, the epidermal thickness was ranked from thin to thick as follows: Naïve>Daviobet>AR100-RA1-Cys>AR100-RA1>Zerumbone>IMQ. Furthermore, the HE-stained sections revealed that comparing to the Naïve group, the IMQ-induced group exhibited increased dermal thickness and a greater number of infiltrating immune cells (indicated by the dark purple-stained nuclei).

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

### REFERENCES

Chopra R, Silverberg JI. Assessing the severity of atopic dermatitis in clinical trials and practice. Clin Dermatol. 2018 September-October; 36(5): 606-615. doi: 10.1016/j.clindermatol.2018.05.012. Epub 2018 Jun. 1. PMID: 30217273.
ADDIN EN.REFLIST Luo, D. Q., H. H. Wu, Y. K. Zhao, J. H. Liu and F. Wang (2016). “Original Research: Different imiquimod creams resulting in differential effects for imiquimod-induced psoriatic mouse models.” Exp Biol Med (Maywood) 241(16): 1733-1738.
van der Fits, L., S. Mourits, J. S. Voerman, M. Kant, L. Boon, J. D. Laman, F. Cornelissen, A. M. Mus, E. Florencia, E. P. Prens and E. Lubberts (2009). “Imiquimod-induced psoriasis-like skin inflammation in mice is mediated via the IL-23/IL-17 axis.” J Immunol 182(9): 5836-5845.

1. A compound, named as AR100-RA1-Cys, which has a structure selected from the group consisting of:

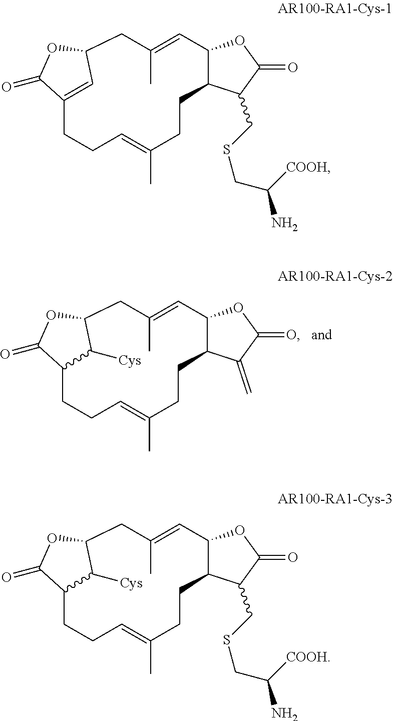

2. The compound AR100-RA1-Cys of claim 1, which is AR100-RA1-Cys-1.

3. The compound AR100-RA1-Cys of claim 1, which is effective in auto-immune disease.

4. A compound, named as AR100-RA1-ST, which has a structure selected from the group consisting of:

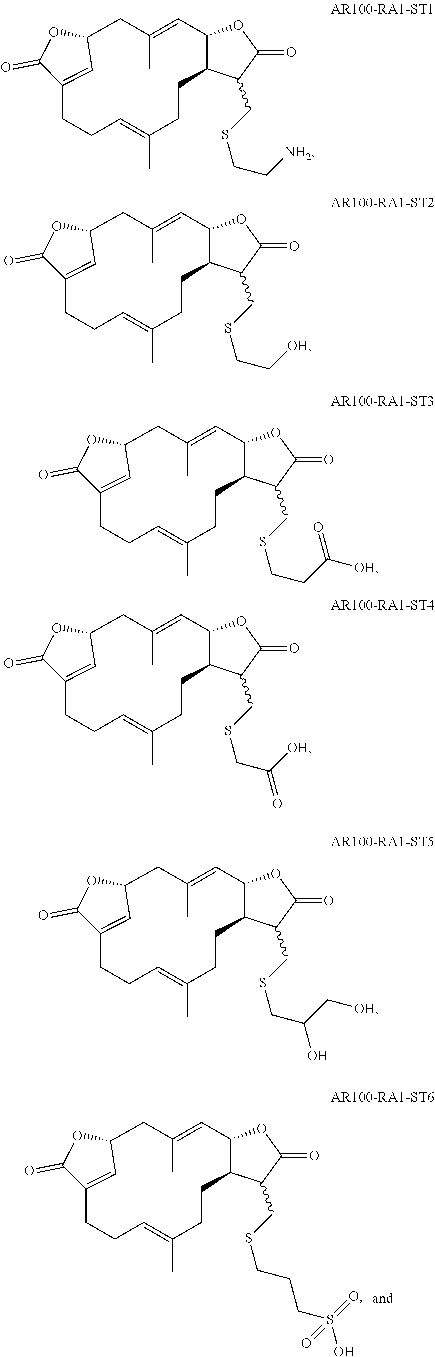

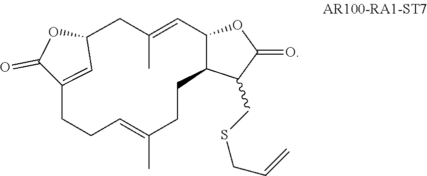

5. The compound AR100-RA1-ST of claim 4, which is AR100-RA1-ST4.

6. The compound AR100-RA1-Cys of claim 4, which is effective in auto-immune disease.

7. The compound according to claim 1, which is effective in treating an auto-immune disease, which auto immune disease is selected from the group consisting of autoimmune hepatitis, psoriasis, autoimmune pancreatitis, Sjogren' syndrome, ulcerative colitis, Crohn's disease, reflex sympathetic dystrophy, post myocardial infarction syndrome, rheumatoid rhinitis, multiple sclerosis, and cardiomyopathy.

8. A method for preparing the compound AR100-RA1-Cys according to claim 1, comprising the steps of:
(A) mixing AR100-RA1 and NaHCO₃ in 75% Ethanol to form a mixture;
(B) adding L-Cysteine Hydrochloride Monohydrate (Cys) into the mixture of step (A);
(C) stirring the mixture of step (B) at around 65° C.;
(D) cooling down the mixture of step (C) to room temperature;
(E) adding HCl into the mixture of step (D) slowly to quench;
(F) filtering and concentrating the mixture of step (E) under reduced pressure; and
(G) purifying the mixture of step (F) to provide AR100-RA1-Cys as light-yellow solid.

9. A method for preparing the compound AR100-RA1-ST according to claim 4, comprising the steps of:
(A) mixing AR100-RA1 and NaHCO₃ in 75% Ethanol to form a mixture;
(B) adding one of the 7 compounds as shown below into the mixture of step (A): 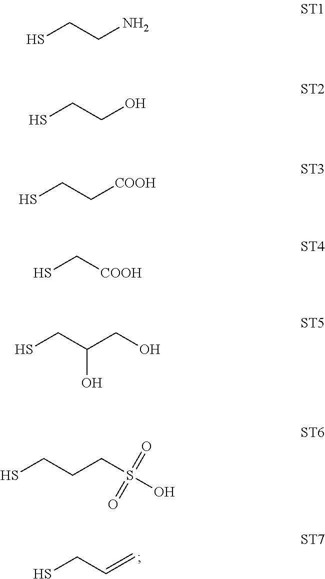
(C) stirring the mixture of step (B) overnight at room temperature;
(D) diluting the mixture of step (C) with water;
(E) adjusting the mixture of step (D) to pH of 2˜4 by adding acid; and
(F) filtering and concentrating the mixture of step (E) to provide AR100-RA1-ST as light-yellow solid.

10. A pharmaceutical composition, which comprises a therapeutically effective amount of the compound as defined in claim 1, or a combination thereof, together with a pharmaceutically acceptable carrier.

11. The pharmaceutical composition of claim 10, which is used for treating an auto-immune disease, wherein the auto-immune disease is selected from the group consisting of autoimmune hepatitis, autoimmune pancreatitis, celiac disease, diabetes mellitus type 1, Sjogren' syndrome, ulcerative colitis, Crohn's disease, reflex sympathetic dystrophy, post myocardial infarction syndrome, Graves' disease, inflammatory bowel disease, multiple sclerosis, psoriasis, rheumatoid arthritis, cardiomyopathy and systemic lupus erythematosus.

12. A method for treating an auto-immune disease in a subject comprising administering to the subject a therapeutically effective amount of the pharmaceutical composition set forth in claim 10.

13. The method of claim 12, wherein the auto-immune disease is selected from the group consisting of autoimmune hepatitis, autoimmune pancreatitis, celiac disease, diabetes mellitus type 1, Sjogren' syndrome, ulcerative colitis, Crohn's disease, reflex sympathetic dystrophy, post myocardial infarction syndrome, Graves' disease, inflammatory bowel disease, multiple sclerosis, psoriasis, rheumatoid arthritis, cardiomyopathy and systemic lupus erythematosus.

14. The method of claim 12, wherein the auto-immune disease is autoimmune hepatitis or psoriasis.

15. The method of claim 12, wherein the subject is human or animals.
